(19) United States
(12) Patent Application Publication
(10) Pub. No.: US 2026/0298735 A1
(43) Pub. Date: Oct. 1, 2026

(54) REACTION FORCE MEASURING PLATE AND REACTION FORCE MEASURING SYSTEM

(71) Applicant: CONTITECH DEUTSCHLAND GMBH, Hannover (DE)

(72) Inventors: Max Konstantin WERHAHN, Hannover (DE); Peter Michael MARIENFELD, Hannover (DE)

(21) Appl. No.: 19/474,786

(22) PCT Filed: Mar. 27, 2024
(86) PCT No.: PCT/EP2024/058222

§ 371 Date: Oct. 13, 2025

(30) Foreign Application Priority Data
Apr. 13, 2023 (DE) 10 2023 203 353.4

(51) Int. Cl. G01L 1/20; A01K 29/00; G01L 1/22; G01L 5/00

(52) U.S. Cl. CPC G01L 1/205; A01K 29/005; G01L 1/2237; G01L 1/2287; G01L 5/0038

(57) ABSTRACT

The present invention relates to a reaction force measuring plate (1) for detecting the distribution of ground reaction forces across the ground contact area of the foot of a hoofed animal or the foot of a human being when it strikes the ground, comprising a preferably rigid carrier plate (3) with a first surface area (3a) facing the ground during use and an opposite second surface area (3b) facing the hoof or foot, or vice versa, a plurality of flat force measuring sensors (5) fixed in position on the first surface area (3a) of the carrier plate (3), a force transmission surface element (11), preferably elastomeric, which is fixed in position to the force measuring sensors (5) parallel to the carrier plate (3) and facing away from the first surface area (3a), and a plurality of elastic force transmission studs (7), in particular a plurality corresponding to the plurality of force measuring sensors, which are fixed to the free surface of the force transmission surface element (11) directly opposite exactly one of the force measuring sensors (5).

The invention relates to a reaction force measuring plate which especially, is intended for use in a hoof boot, a hoof or a shoe and is suitable for determining a flat reaction force distribution when the hoof of a hoofed animal or the foot of a human strikes the ground. It also relates to a reaction force measuring system comprising such a measuring plate, and in addition, a horseshoe, hoof boot or shoe equipped with such a measuring plate is also claimed.

For the gait and health analysis of a horse or other hoofed animal (e.g. camel), it is desirable to measure the reaction forces generated when it strikes the ground, not only at a single point, but with a certain displacement across the entire contact area of the hoof. The measured values should enable conclusions to be drawn about the animal's state of health, e.g. with regard to lameness or strain on the musculoskeletal system.

While there are a whole range of solutions that are available for corresponding tasks in the fields of medical diagnostics, training condition analysis and rehabilitation in humans, the availability of corresponding systems for hoofed animals is limited. There is a commercially available product from Tekscan and other systems with a similar purpose, however these are based on measuring acceleration rather than reaction forces.

GB 2 482 192 B discloses the attachment of force sensors to a horseshoe for such purposes and the local storage of their signals and/or their transmission via a wireless transmitter to a remote receiving and evaluation station.

According to DE 10 2011 016 344 A1, force sensors are used in an elastomer body for a corresponding purpose. The elastomer body, which in turn is to be inserted into a hoof boot, is designed to enable the use of commercially available resistive force sensors with their limited force measuring range for reaction force analysis in horses with their relatively high ground pressure.

From US 2020/319044 A1 we learn of another system for the area-dissipated recording of reaction forces on the hooves of a hoofed animal, which transmits the sensor signals via a wireless transmitter to a remote evaluation unit. This system has a complex design consisting of a base plate and a ground plate, which are guided precisely in relation to each other and between which several force application cylinders and a plurality of resistive force measuring sensors are housed, as well as a microprocessor unit, a communication unit and a battery to supply force to the components. The function of the ‘force application cylinders’ is not apparent from the printed material, nor is the method of attachment to the hoof.

DE 10 2021 211 795.3 (unpublished) describes a reaction force measuring plate for recording the distribution of ground reaction forces across the ground contact area of the foot of a hoofed animal or the foot of a human being when it strikes the ground, with a rigid carrier plate with a first surface facing the ground during use and a second surface facing the hoof or foot, a plurality of flat force measuring sensors fixed in position on the first surface of the carrier plate, and several elastic force transmission studs, especially a plurality corresponding to the plurality of force measuring sensors, fixed to the free surfaces of the force measuring sensors.

A disadvantage of the known possibilities for measuring the force exerted by the foot of a hoofed animal or the foot of a human being when it strikes the ground, i.e. when it comes into contact with the ground, is that only the distributions of force can be measured, however no total forces occur, as shunt forces occur in addition to the discrete sensor positions.

A disadvantage of using individual force transmission studs for each force measuring sensor of the reaction force measuring plate of DE 10 2021 211 795.3 is that even a slight misalignment between the force transmission studs and the force measuring sensor during operation causes the characteristics or calibration curve of the force measuring sensor (measured value vs. applied force) to change. This can result in significant inaccuracies in the measured force.

One task of the present invention is to improve the possibilities for measuring the force exerted by the foot of a hoofed animal or the foot of a human being when it strikes the ground. Especially, a preferably compact sensor plate with discrete sensor positions and the transmission of force into the same is to be created, resulting in stable and robust sensor characteristics. Additionally or alternatively, the transmission of force without a shunt force should be used to enable the measurement of absolute forces and minimize potential errors in the measured force distribution. In any case, this should be done in a way that is as simple, cost-effective, space-saving and/or weight-saving as possible. At the very least, an alternative to the familiar options should be created.

According to the invention, the task is solved by a reaction force measuring plate, a reaction force measuring system and a hoof boot, horseshoe or shoe with the features of the independent patent claims. Advantageous further embodiments are described in the subclaims.

The present invention therefore relates to a reaction force measuring plate for detecting the distribution of ground reaction forces across the ground contact area of the foot of a hoofed animal or the foot of a human being when it strikes the ground, with a preferably rigid carrier plate with a first surface area facing the ground during use and an opposite second surface facing the hoof or foot, or vice versa, a plurality of flat force measuring sensors fixed in position on the first surface area of the carrier plate, a preferably elastomeric force transmission surface element which is fixed in position parallel to the carrier plate and facing away from the first surface area on the force measuring sensors, and several, in particular a plurality corresponding to the plurality of force measuring sensors, elastic force transmission studs which are fixed on the free surface of the force transmission surface element directly opposite exactly one of the force measuring sensors. The ground is a substrate that can basically be of any composition or made of any material.

The reaction force measuring plate according to the invention may have a carrier plate that is substantially both flexurally rigid and incompressible (but possibly elastically flexible). Such a reaction force measuring plate has a simple design and is therefore inexpensive to manufacture, robust in practical use, and easy to handle. It also enables a sufficiently resolved recording of the reaction forces across the surface of the hoof with low leakage of the force and therefore high accuracy. It is also flat and lightweight in terms of structure, making it not only easy to use but also easy to transport.

The present invention is based on the realization that the force transmission studs, which are each arranged directly opposite one of the force measuring sensors, can be used to transmit the ground contact forces to the respective force sensors, as described in DE 10 2021 211 795.3. In addition, however, the fact that a force transmission surface element accommodates the force transmission studs and thus positions them relative to each other and to the force measuring sensors means that any misalignment between the force transmission studs and the force measuring sensor can be avoided during operation, or at least sufficiently reduced in order to avoid and/or at least sufficiently reduce a change and/or an influencing of the characteristics and/or the calibration characteristic curve in order to be able to measure the forces with sufficient accuracy.

The force transmission surface element can also be called a pressure conduit element. In any case, the force transmission surface element can be designed as a thin flat plane, which can preferably be bonded to a carrier film by material engagement, especially by vulcanization, as will be described in more detail below.

The force transmission surface element can also serve as a thin protective layer and, for this purpose, be designed in such a way, that the force transmission surface element covers and protects at least most of the force measuring sensors or their sensor carrier film.

Preferably, at least five force measuring sensors can be used. Preferably, at least, and particularly preferably exactly seven force measuring sensors can be used, which can be arranged along the edge. In any case, the force measuring sensors can be arranged at approximately equal distances from each other in the circumferential direction. This can enable representative recording of force values with limited cost.

As explicitly stated and described above, the carrier plate can be used with the first surface facing the ground and the second opposite surface facing the hoof or foot. However, the arrangement can also be reversed, with the carrier plate facing the hoof or foot with the first surface in use and facing the ground with the second opposite surface. This can increase the utility and design possibilities.

Especially, the arrangement of the carrier plate with the first surface facing the hoof or foot during use is advantageous because the force measuring sensors and elastic force transmission studs then act against the hoof. Accordingly, the carrier plate does not need to be connected directly to the hoof or horseshoe. Instead, the fixation can be achieved, for example, by means of a hoof boot, which can also be formed by a protective element of the reaction force measuring plate itself, as will be described in more detail below.

According to one aspect of the invention, the force transmission studs and/or the force transmission surface elements are formed of an elastomeric material. This can cause and/or enable elastic force transmission. Especially, the hardness of the elastomer or elastomeric material of the force transmission studs and/or the force transmission surface element can be between 30 and 85 ShA.

According to a further aspect of the invention, the force transmission studs are formed integrally with the force transmission surface element. Thus, the force transmission studs and the force transmission surface element are formed integrally. This can be done using the same material or different materials, although the latter can increase both the manufacturing costs and the design options.

According to a further aspect of the invention, the force transmission studs have a linear or trapezoidal contact surface with the ground and/or with the respective force measuring sensor. Preferably, a linear or elongated contact surface on the side facing away from the force measuring sensor and a larger, wider contact surface in the direction of the force measuring sensor are to be preferred. This can be achieved, for example, by using a trapezoidal shape, but also by using a straight-line design for the height of the force transmission studs. In any case, this can affect force transmission.

According to a further aspect of the invention, the force transmission surface element is bonded to the force transmission studs with the force measuring sensors or a sensor carrier film facing away from the force transmission studs. This can represent a connection option that can be made directly between force measuring sensors and force transmission surface elements. If the force measuring sensors are arranged on a sensor carrier film, and especially, printed on it, as will be described in more detail below, the sensor carrier film can also be covered and protected by means of the force transmission surface element.

According to a further aspect of the invention, the force transmission surface element is vulcanized onto a carrier film away from the force transmission studs, and the carrier film is bonded to the force measuring sensors or a sensor carrier film. The carrier film can thus be used to create a surface that improves the adhesive effect on the force measuring sensors and/or a sensor carrier film. The carrier film may preferably consist of a material chemically similar to the sensor carrier film in order to enable or promote a material bond with the sensor carrier film. In any case, the carrier film can preferably consist of a thermoplastic or elastomeric material (e.g., PET, TPE, NR, EPDM), which can be bonded to the sensor carrier film, preferably in a vulcanization process or in an injection molding process.

According to a further aspect of the invention, the carrier plate has the shape of a closed horseshoe, circular ring, U, or polygon, in particular with a recess in the center area. This allows adaptation for use with various hoofed animals or other vertebrates (including humans).

According to a further aspect of the invention, the force measuring sensors, together with associated sensor signal lines and optional power supply lines, are implemented on a continuous sensor carrier film, which is fixed in particular to the first surface area of the carrier plate. This can simplify implementation and manufacture.

According to a further aspect of the invention, the force measuring sensors or the sensor carrier films are bonded to the carrier plate. This can be an easily implementable embodiment. Alternatively, the force measuring sensors or the sensor carrier film are detachably attached to the carrier plate, in particular inserted into suitable guides or locked together. It goes without saying that such solutions are more complex to construct and potentially more susceptible to failure, so they are more likely to be suitable for special applications.

According to a further aspect of the invention, the effective area of the force measuring sensors is in the range between 0.5 cm² and 10 cm², in particular between 2 cm² and 5 cm². It is understood that when using a relatively large number of force measuring sensors, and especially in configurations of the measuring plate provided for animals with relatively small hoof or foot areas, the effective area may be relatively small, whereas in configurations with a relatively small number of sensors and for animals with large detection areas, it may be closer to or even above the preferred upper limit.

According to a further aspect of the invention, all force measuring sensors are substantially rectangular in shape and have the same geometric shape and effective area. This can facilitate the technologically easy and cost-effective manufacture of the sensors and the configurability of different embodiments of the measuring plate.

According to a further aspect of the invention, the force measuring sensors are resistive dielectric sensors, which in particular comprise a first conductive layer, a dielectric layer on top of this, which is surrounded and delimited by a spacer determining the shape of the force measuring sensor, and a second conductive layer on top of the dielectric layer and the spacer. In addition to the structure mentioned here as a variant, such resistive force sensors can also have a different, i.e., known structure. In principle, piezoelectric, capacitive, or inductive sensors or electroactive polymers can also be used in the reaction force measuring plate in addition to resistive dielectric sensors.

According to another aspect of the invention, the carrier plate consists of organic sheet metal, spring steel, or plastic.

The present invention also relates to a reaction force measuring system with a reaction force measuring plate as described above and a wireless sensor signal transmission unit attached to it and connected to the force measuring sensors in terms of signals, especially according to the Bluetooth standard, as well as a sensor signal receiver, evaluation and display device arranged remotely from the reaction force measuring plate, which comprises a wireless sensor signal receiving unit communicating with the reaction force measuring plate via the sensor signal transmission unit.

Optionally, a carrier plate may also be provided with a device for preprocessing sensor signals, for example, to facilitate transmission via the transmission unit.

Provided that the force measuring sensors are implemented on a sensor carrier film with corresponding conductors, the transmission unit can also be arranged on this sensor carrier film and connected to the sensors via the conductors.

The present invention also relates to a hoof boot, horseshoe, or shoe to which a reaction force measuring plate as described above is fixed. Ultimately, this proposes a horseshoe or hoof boot (for use on hoofed animals) or a shoe (for use on humans) that is equipped with a reaction force measuring plate on the underside. In the case of hoof boots, depending on their specific design, the measuring plate can be positioned on the inside and, if necessary, fixed to the hoof or horseshoe. Although the invention is primarily intended for use on hoofed animals, it is also conceivable that the reaction force measuring plate according to the invention could be used for certain examinations on humans, for example to obtain information for rehabilitation or training purposes. This person will then wear shoes equipped with the measuring plate according to the invention.

The advantages and practicalities of the invention are also apparent from the description of embodiments based on the figures. These show:

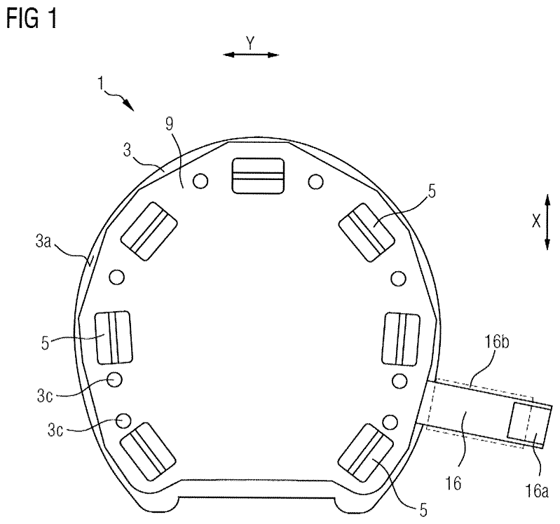

FIG. 1 the structure of an example reaction force measuring plate from a perspective view from below;

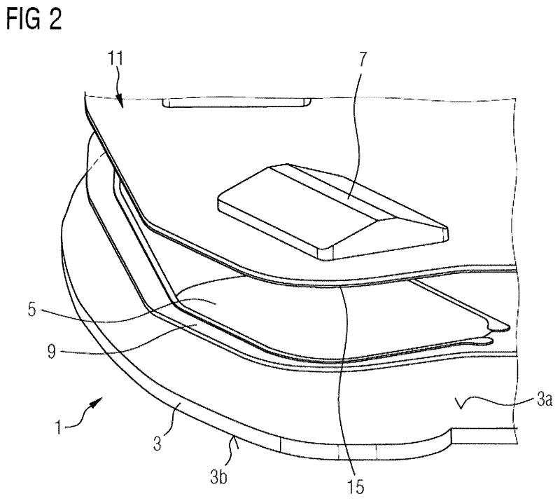

FIG. 2 a perspective view of a section of FIG. 1 as an exploded view;

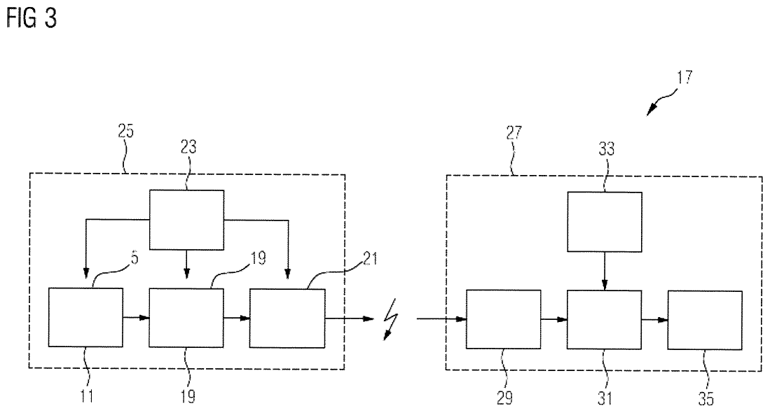

FIG. 3 a schematic representation of the attachment of the reaction force measuring plate according to the invention to a horse's hoof; and

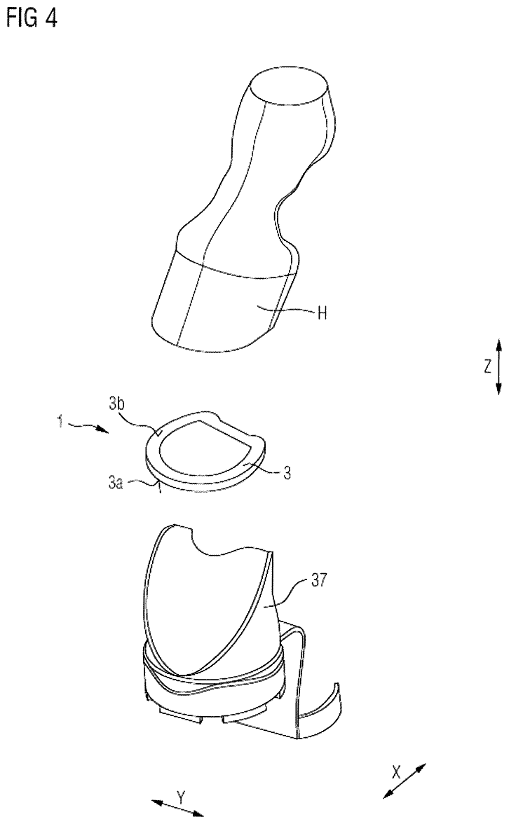

FIG. 4 a schematic diagram of an embodiment of the reaction force measuring system according to the invention, in the form of a block diagram.

The above figures are described in Cartesian coordinates with a longitudinal direction X, a transverse direction Y perpendicular to the longitudinal direction X, and a vertical direction Z perpendicular to both the longitudinal direction X and the transverse direction Y. The longitudinal direction X can also be referred to as depth X, the transverse direction Y as width Y, and the vertical direction Z as height Z. The longitudinal direction X and the transverse direction Y together form the horizontal X, Y, which can also be referred to as the horizontal plane X, Y. The longitudinal direction X, the transverse direction Y, and the vertical direction Z can also be referred to collectively as spatial directions X, Y, Z or as Cartesian spatial directions X, Y, Z.

FIG. 1 shows, in a perspective view from below, the structure of an exemplary reaction force measuring plate 1 with a closed horseshoe-shaped rigid carrier plate 3, which has a first surface 3a and a second surface 3b. Seven resistive force measuring sensors 5 with a matching rectangular basic shape are attached to the first surface area 3a at equal distances from each other. The free surface of each of the force measuring sensors 5 faces downward toward a ground surface (not shown) and thus away from, for example, the horse's hoof H. The force measuring sensors 5 can also be referred to as force sensors 5 or force sensor elements 5.

The force measuring sensors 5 are integrally implemented on a sensor carrier film 9, which also carries sensor signal lines as conductors (not shown) for connecting each sensor. The sensor carrier film 9, together with the force measuring sensors 5 formed thereon, can be manufactured using conventional means of printed circuit board technology, including printed electronics, which are known to those skilled in the art. The sensor carrier film 9 is applied with its rear side to the first surface 3a of the carrier 3. This is done using an adhesive and/or an adhesive layer, such as double-sided adhesive tape.

There is also an elastomeric force transmission surface element 11 which, like the sensor carrier film 9, corresponds approximately to the surface area of the rigid carrier plate 3. The elastomeric force transmission surface element 11 can also be referred to as an elastomeric pressure conduit element 11. The elastomeric force transmission surface element 11 has a carrier film 15 facing the rigid carrier plate 3 or the sensor carrier film 9, which is connected to the force transmission surface element 11 by vulcanization and also corresponds approximately to the surface area of the rigid carrier plate 3. The force transmission surface element 11 is also bonded to the sensor carrier film 9 by means of the carrier film 15, which is facilitated or achieved by the corresponding material combination. At the same time, the vulcanized bond between the carrier film 15 and the force transmission surface element 11 allows an elastomeric material to be used for the force transmission surface element 11 and yet still be bonded to the sensor carrier film 9 by means of the carrier film 15.

Several elastomeric force transmission studs 7, which can also be referred to as elastomeric pressure conduit studs 7, are formed on the elastomeric force transmission surface element 11, which point toward the substrate or away from the force measuring sensors 5 or the sensor carrier film 9. Each force measuring sensor 5 is assigned exactly one force transmission stud 7, so that the force measuring sensor 5 and its force transmission stud 7 are designed to be congruent and positioned relative to each other. This positioning is ensured by the fact that the force transmission studs 7 are formed integrally with the elastomeric force transmission surface element 11.

In order to connect the reaction force measuring plate 1 to the hoof of the vertebrate or to any additional element located between them, the carrier plate in the embodiment shown has holes 3c (shown only in FIG. 1) into which, for example, hoof nails can be driven or a screw can be inserted.

The force measuring sensors 5 are connected via printed sensor signal lines and can be read out, as will be described in more detail below. For this purpose, a connector element 16 in the form of a plug outlet 16 is provided, which ends in a plug 16a. The plug outlet 16 and an internal part of the plug 16a are enclosed by a cable guard 16b, thereby protecting them from external influences.

FIG. 3 is a schematic diagram of a reaction force measuring system 17, which can be formed, especially with a reaction force measuring plate according to one of FIGS. 1 to 2. The representation takes the form of a functional block diagram and is not intended to show the exact design of the system components.

In addition to the force measuring sensors 5 and their sensor signal lines shown in FIGS. 1 and 2, the reaction force measuring system 17 also comprises a sensor signal pre-processing unit 19, which is connected to the force measuring sensors 5 via the sensor signal lines and serves to perform preprocessing and formatting of the sensor signals that is advantageous for external signal transmission. On the output side, the sensor signal pre-processing unit 19 is connected to a wireless sensor signal transmitter 21, which can operate according to the Bluetooth standard, for example, but also according to another standard for wireless message transmission, depending on the application. The above-mentioned components are assigned an energy source 23, which in the simplest case is a commercially available primary cell or a rechargeable battery. If a rechargeable battery is used, it can be assigned a charging socket (not shown in the figure) for recharging when installed.

All the above-mentioned components are advantageously arranged on the reaction force measuring plate 1, and especially protected in its interior, and are referred to here as hoof component 25. Specifically, the functional units of the hoof component 25 can all be realized on the sensor carrier film 9.

When the system is in use, the hoof component 25 is in wireless signal connection via the sensor signal transmitter 21 with a sensor signal receiver, evaluation and display device 27, which is placed away from the animal (or human) to be examined. The device 27 comprises a wireless sensor signal receiver 29 which is configured to communicate with the sensor signal transmitter 21 on the hoof component 25 and which feeds the received signals to a signal evaluation unit 31, where they are subjected to evaluation according to a program stored in a program memory 33.

Finally, a display unit 35 serves to present the evaluation results, for example to a therapist or trainer. The components of the sensor signal receiver, evaluation and display device can be implemented, for example, in a notebook, tablet or smartphone with a suitable evaluation app.

FIG. 4 shows an example of a possible use of the reaction force measuring plate 1 according to the invention, placed inside a hoof boot 37 of a commercially available type, which is attached to a hoof H of a horse. In the example shown, the reaction force measuring plate 1—depending on the design of the hoof boot—is temporarily fixed to the foot with a suitable, removable adhesive and/or bonding agent and then the hoof boot 37 is slipped over it, or the reaction force measuring plate 1 is inserted into the hoof boot and the hoof boot is then fixed to the hoof.

The implementation of the invention is not limited to the examples and aspects mentioned above, but is also possible in a variety of modifications that are within the scope of the appended claims.

### REFERENCE LIST (PART OF THE DESCRIPTION)

H hoof and/or horse hoof
X longitudinal direction; depth
Y transverse direction; width
Z vertical direction; height
X, Y horizontal; horizontal plane
1 reaction force measuring plate
3 carrier plate
3a first surface
3b second surface
3c openings
5 force measuring sensors; force sensors; force measuring sensor elements
7 (elastomeric) force transmission studs; (elastomeric) pressure conduit studs
9 sensor carrier film
11 (elastomeric) force transmission surface element; (elastomeric) pressure conduit element
15 carrier film
16 connector element; plug outlet
16a plug
16b cable guard
17 reaction force measuring system
19 sensor signal pre-processing unit
21 wireless sensor signal transmitter
23 energy source
25 hoof component
27 sensor signal, sensor signal receiver, evaluation and display device
29 wireless sensor signal receiver
31 signal evaluation unit
33 program memory
35 display unit
37 hoof boot

1. A reaction force measuring plate for recording the distribution of ground reaction forces over the ground contact area of the foot of a hoofed animal or the foot of a human when it strikes the ground, with
a preferably rigid carrier plate with a first surface area facing the ground during use and an opposite second surface area facing the hoof or foot, or vice versa,
a plurality of flat force measuring sensors fixed in position on the first surface of the carrier plate,
a force transmission surface element, preferably elastomeric, which is fixed in position on the force measuring sensors parallel to the carrier plate and facing away from the first surface area, and
a plurality of elastic force transmission studs, in particular a plurality corresponding to the plurality of force measuring sensors, which are fixed to the free surface of the force transmission surface element directly opposite exactly one of the force measuring sensors.

2. The reaction force measuring plate according to claim 1,
wherein the force transmission studs and/or the force transmission surface element are formed of an elastomeric material.

3. The reaction force measuring plate according to claim 1,
wherein the force transmission studs are formed integrally with the force transmission surface element.

4. The reaction force measuring plate according to claim 1,
wherein the force transmission studs have a linear or trapezoidal contact surface with the ground and/or with the respective force measuring sensor.

5. The reaction force measuring plate according to claim 1,
wherein the force transmission surface element is bonded to the force measuring sensors (5) or a sensor carrier film (9) away from the force transmission studs.

6. The reaction force measuring plate according to claim 1,
wherein the force transmission surface element is vulcanized on a carrier film facing away from the force transmission stud, and the carrier film is bonded to the force measuring sensors or a sensor carrier film.

7. The reaction force measuring plate according to claim 1,
wherein the carrier plate has the shape of a closed horseshoe, circular ring or U or polygon, especially with a recess in the central area.

8. The reaction force measuring plate according to claim 1,
wherein the force measuring sensors together with associated sensor signal lines and optionally power supply lines are realized on a continuous sensor carrier film which is fixed in particular on the first surface of the carrier plate.

9. The reaction force measuring plate according to claim 1,
wherein the force measuring sensors or the sensor carrier film bonded to the carrier plate, or
wherein the force measuring sensors or the sensor carrier films are detachably fastened to the carrier plate, in particular inserted into suitable guides.

10. The reaction force measuring plate according to claim 1,
wherein the effective area of the force measuring sensors is in the range between 0.5 cm² and 10 cm², in particular 2 cm² and 5 cm².

11. The reaction force measuring plate according to claim 1,
wherein all force measuring sensors are substantially rectangular in shape and have the same geometric shape and effective area.

12. The reaction force measuring plate according to claim 1,
wherein the force measuring sensors are resistive-dielectric sensors, which in particular comprise a first conductive layer, on this a dielectric layer which is surrounded and delimited by a spacer determining the shape of the force measuring sensor, and on the dielectric layer and the spacer a second conductive layer.

13. The reaction force measuring plate according to claim 1,
wherein the carrier plate consists of organic sheet metal, spring steel, or plastic.

14. A reaction force measuring system, with
a reaction force measuring plate according to claim 1 and
a wireless sensor signal transmission unit attached to it
and connected by signals to the force measuring sensors, especially according to the Bluetooth standard, as well as
a sensor signal receiver, evaluation and display device arranged remotely from the reaction force measuring plate, which comprises a wireless sensor signal receiving unit communicating with the sensor signal transmission unit on the reaction force measuring plate.

15. A hoof boot, horseshoe, or shoe to which a reaction force measuring plate according to claim 1.
